(19) United States
(12) Patent Application Publication
(10) Pub. No.: US 2014/0005160 A1
(43) Pub. Date: Jan. 2, 2014

(54) Method for Treating Refractory Cancer

(71) Applicants: Hooshmand Sheshbaradaran, Matawan, NJ (US); Rebecca Baerga, Matawan, NJ (US); Jenel Cobb, Matawan, NJ (US)

(72) Inventors: Hooshmand Sheshbaradaran, Matawan, NJ (US); Rebecca Baerga, Matawan, NJ (US); Jenel Cobb, Matawan, NJ (US)

(21) Appl. No.: 13/991,358

(22) PCT Filed: Dec. 1, 2011
(86) PCT No.: PCT/US11/62746

§ 371 Date: Aug. 21, 2013

Related U.S. Application Data
(60) Provisional application No. 61/418,836, filed on Dec. 1, 2010.

(51) Int. Cl. A61K 31/555

(52) U.S. Cl. CPC A61K 31/555
USPC 514/187

(57) ABSTRACT

Therapeutic methods for treating refractory cancers are disclosed comprising administering to a patient in need of treatment a compound of Formula (I) disclosed herein.

### CROSS-REFERENCE TO RELATED U.S. APPLICATIONS

This application claims the benefit of U.S. Provisional Application No. 61/418,836 filed on Dec. 1, 2010, the content of which is incorporated herein by reference.

### FIELD OF THE INVENTION

The present invention generally relates to methods for treating cancer, and particularly to a method of treating refractory cancer.

### BACKGROUND OF THE INVENTION

Tris(8-quinolinolato)gallium(III) is an organic gallium complex that has been suggested to be useful in certain types of cancer. For example, U.S. Pat. No. 7,919,486 discloses and claims the use of tris(8-quinolinolato)gallium(III) and related compounds for the treatment of melanoma.

### SUMMARY OF THE INVENTION

It has now been discovered that the compound tris(8-quinolinolato)gallium(III) can be especially effective in treating selected refractory cancers. Specifically, it has been discovered that the compound tris(8-quinolinolato)gallium(III) is effective in inducing apoptosis in certain tested cancer cell lines resistant to selected anti-cancer drugs, and thus can be useful in treating cancer resistant to, or recurring after prior treatment of, such anti-cancer drugs.

Accordingly, in a first aspect, the present invention provides a method of treating selected refractory cancer as described below with a therapeutically effective amount of a compound according to Formula (I) below or a pharmaceutically acceptable salt thereof (e.g., tris(8-quinolinolato)gallium(III)).

In a second aspect, the present invention provides a method of preventing or delaying the onset of selected refractory cancer, comprising administering to a patient having a cancer previously treated with one or more anti-cancer drugs, a prophylatically effective amount of a compound according to Formula (I) below or a pharmaceutically acceptable salt thereof (e.g., tris(8-quinolinolato)gallium(III)).

The present invention further provides use of a compound according to Formula (I) below or a pharmaceutically acceptable salt thereof (e.g., tris(8-quinolinolato)gallium(III)) for the manufacture of a medicament useful for treating, preventing or delaying the onset of selected refractory cancer.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### DETAILED DESCRIPTION OF THE INVENTION

The present invention generally provides methods for treating certain refractory cancers. The term “refractory to (a drug),” as used herein, means that a particular cancer either has failed to respond favorably to a specific anti-neoplastic treatment, or alternatively, recurs or relapses after responding favorably to a specific anti-neoplastic treatment. Accordingly, for example, a non-small cell lung cancer “refractory to” erlotinib means that a non-small cell lung cancer either has failed to respond favorably to, or has exhibited resistance to, a treatment regimen that includes, but not necessarily limited to, erlotinib, or alternatively, has recurred or relapsed after responding favorably to the treatment regimen.

To identify refractory cancer, patients undergoing chemotherapy treatment can be carefully monitored for signs of resistance, non-responsiveness or recurring cancer. This can be accomplished by monitoring the patient's cancer's response to a chemotherapy treatment. The response, lack of response, or relapse of the cancer to the treatment can be determined by any suitable method practiced in the art. For example, this can be accomplished by the assessment of tumor size and number. An increase in tumor size or, alternatively, tumor number, indicates that the tumor is not responding to the chemotherapy, or that a relapse has occurred. The determination can be done according to the “RECIST” criteria as described in detail in Therasse et al, J. Natl. Cancer Inst., 92:205-216 (2000).

In accordance with the present invention, a method is provided for treating, preventing, or delaying the onset of, selected refractory cancer with a therapeutically effective amount of a gallium complex of Formula (I)

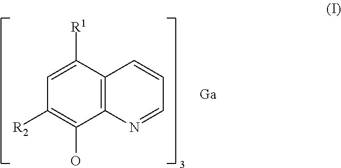

wherein R¹ represents hydrogen, a halogen or a sulfono group SO₃M, in which M is a metal ion, and R² represents hydrogen, or R¹ is Cl and R² is I, or a pharmaceutically acceptable salt thereof. That is, the present invention is directed to the use of a compound according to Formula (I) or a pharmaceutically acceptable salt thereof. Examples of pharmaceutically acceptable salts include alkali metal salts (e.g., sodium or potassium salt), ammonium salts, etc. In preferred embodiments, the compound tris(8-quinolinolato)gallium(III) is used in the method. Tris(8-quinolinolato)gallium(III), also known as tris-(8-hydroxyquinoline)gallium, is a gallium complex compound first made by Professor Bernhard Keppler and is disclosed in, e.g., U.S. Pat. No. 5,525,598.

In accordance with one aspect of the present invention, a method is provided for treating a cancer previously treated with a treatment regimen comprising one or more drugs chosen from the group consisting of 5-FU and prodrugs thereof (e.g., capecitabine, tegafur and S1), camptothecins, gefitinib, erlotinib, temsirolimus and temozolomide. The method comprises treating such a previously treated cancer with a therapeutically effective amount of a compound according to Formula (I) or a pharmaceutically acceptable salt thereof (e.g., tris(8-quinolinolato)gallium(III)). In some embodiments, a patient is identified as having a refractory cancer previously treated with a treatment regimen comprising one or more of the above-named anti-cancer drugs. The patient may have failed to respond to such a treatment regimen or have a cancer relapsing or recurring after responding favorably to the treatment regimen. In some specific embodiments, the refractory cancer treated is breast cancer, colon cancer, lung cancer (NSCLC or SCLC), prostate cancer, renal cancer (renal cell carcinoma) or melanoma.

In another aspect, a method is provided for treating a cancer having a tumor tissue or cell overexpressing the BCRP protein. In some specific embodiments, the refractory cancer treated is breast cancer, colon cancer, prostate cancer, melanoma or renal cancer. For this purpose, tumor cell or tissue samples can be obtained from a patient and analyzed for the expression of BCRP according to well-known techniques in the art. A patient identified as having a tumor overexpressing BCRP is then treated with a therapeutically effective amount of a compound according to Formula (I) or a pharmaceutically acceptable salt thereof (e.g., tris(8-quinolinolato)gallium(III)).

1. In one embodiment, the method of the present invention is used for treating a patient for a colorectal cancer previously treated with a treatment regimen comprising oxaliplatin and/or 5-FU (or a prodrug thereof such as capecitabine, tegafur and S1). In some specific embodiments, the colorectal cancer is refractory or resistant to oxaliplatin and/or 5-FU (or a prodrug thereof such as capecitabine, tegafur and S1). That is, a patient having colorectal cancer (primary or metastatic) previously treated with a treatment regimen including oxaliplatin and/or 5-FU (or a prodrug thereof such as capecitabine, tegafur and S1) is identified. Such a patient may have not responded to such a treatment regimen, or have a relapsing or recurring colorectal cancer after such a treatment regimen. To illustrate, the treatment regimen including oxaliplatin and/or 5-FU (or a prodrug thereof such as capecitabine, tegafur and S1) may be a regimen of 5-FU (or capecitabine) alone or in combination with leucovorin, or FOLFOX or FOLFIRI with or without bevacizumab. Once such a patient is identified, the patient can then be treated with a therapeutically effective amount of a gallium complex of Formula (I) or a pharmaceutically acceptable salt thereof, e.g., tris(8-quinolinolato)gallium(III). The refractory colorectal cancer can be at any stage, either local or metastatic.

In yet another embodiment, the method of the present invention is used for treating breast cancer previously treated with a treatment regimen comprising one or more drugs chosen from the group of cisplatin, 5-FU (or a prodrug thereof such as capecitabine, tegafur and S1), vincristine, vinblastine, vinorelbine, paclitaxel, docetaxel, etoposide, mitoxantrone, doxorubincin, epirubicin, topotecan, irinotecan, methotrexate, camptothecins, gefitinib and imatinib. In some embodiments, the patient's breast cancer may be refractory or resistant to one or more of the above-named anti-cancer drugs. In specific embodiments, the method is used to treat breast cancer refractory or resistant to 5-FU (or a prodrug thereof such as capecitabine, tegafur and S1), paclitaxel, doxorubicin or cisplatin. That is, the method comprises administering a therapeutically effective amount of a gallium complex of Formula (I) or a pharmaceutically acceptable salt thereof, e.g., tris(8-quinolinolato)gallium(III), to a patient identified as having a breast cancer that refractory to one or more of such anti-cancer drugs.

The method of the present invention is also particularly useful in treating lung cancer (e.g., non-small cell lung cancer (NSCLC) or small cell lung cancer (SCLC)) previously treated with a treatment regimen comprising one or more drugs chosen from the group of paclitaxel, and EGFR inhibitors such as erlotinib, gefitinib, and icotinib. In some embodiments, the method is used for treating NSCLC or SCLC refractory to or resistant to one or more drugs chosen from the group of erlotinib, gefitinib and icotinib. The patient either did not respond to such a treatment regimen, or the cancer relapsed or recurred after the treatment regimen. Thus, the method comprises administering to an identified patient in need of the treatment a therapeutically effective amount of a gallium complex of Formula (I) or a pharmaceutically acceptable salt thereof, e.g., tris(8-quinolinolato)gallium(III).

In another embodiment, the present invention provides a method of treating cancer (e.g., NSCLC or colorectal cancer) having an activating mutation in the KRAS gene (e.g., a mutation in codon 12, 13 or 61 of the KRAS gene). The method comprises administering to a cancer patient (e.g., NSCLC or colorectal cancer) identified as having an activating mutation in the KRAS gene (e.g., a mutation in codon 12, 13 or 61 of the KRAS gene) of the tumor cells, a therapeutically effective amount of a gallium complex of Formula (I) or a pharmaceutically acceptable salt thereof, e.g., tris(8-quinolinolato)gallium(III). Optionally, the method comprises a step of identifying a patient (e.g., a non-small cell lung cancer or colorectal cancer patient) whose tumor harbors an activating mutation in the KRAS gene. Somatic activating mutations (e.g., G12C, G12D, G12S, G12V, G13C, etc.) in the KRAS gene in tumors such as lung adenocarcinoma (e.g., NSCLC) and colorectal cancer are well known in the art. Methods of detecting an activating mutations exons 2 and 3 in the KRAS gene are also well-known in the art. See e.g., Pao et al., PLOS Med., 2(1):57-61 (2005).

In yet another embodiment, the method is used to treat prostate cancer previously treated with a treatment regimen including docetaxel, paclitaxel, and/or vinblastine. In some embodiments, the prostate cancer either failed to respond to such a treatment regimen, or relapsed or recurred after the treatment regimen. Thus, a patient suitable for the method of the present invention is identified, and administered with a therapeutically effective amount of a gallium complex of Formula (I) or a pharmaceutically acceptable salt thereof, e.g., tris(8-quinolinolato)gallium(III). For purpose of preventing or delaying the onset of refractory prostate cancer, a patient previously treated with a regimen comprising one or more of docetaxel, paclitaxel, and vinblastine is identified and the patient is subsequently administered with a therapeutically effective amount of a gallium complex of Formula (I) or a pharmaceutically acceptable salt thereof, e.g., tris(8-quinolinolato)gallium(III).

Additionally, the method of the present invention is also useful in treating melanoma previously treated with a regimen comprising temozolomide. In some embodiments, the melanoma is either refractory or resistant to temozolomide. Thus, the method comprises administering to a patient identified as having melanoma previously treated with a regimen comprising temozolomide, a therapeutically effective amount of a gallium complex of Formula (I) or a pharmaceutically acceptable salt thereof, e.g., tris(8-quinolinolato)gallium(III).

The method of the present invention is also useful in treating renal cancer, particularly renal cell carcinoma (RCC) previously treated with a regimen comprising temsirolimus. In some embodiments, the renal cancer is either refractory or resistant to temsirolimus. Thus, the method comprises administering to a patient identified as having renal cancer, particularly renal cell carcinoma, previously treated with a regimen comprising temsirolimus, a therapeutically effective amount of a gallium complex of Formula (I) or a pharmaceutically acceptable salt thereof, e.g., tris(8-quinolinolato)gallium(III).

For purposes of preventing, or delaying the onset of, cancer recurrence, cancer patients who have been treated with a previous treatment regimen as described above in the various embodiments, and who are in remission or in a stable or progression free state may be treated with a prophylactically effective amount a compound of Formula (I) or a pharmaceutically acceptable salt thereof (e.g., tris(8-quinolinolato)gallium(III)), to effectively prevent or delay the recurrence or relapse of the cancer.

As used herein, the phrase “treating . . . with . . . ” or a paraphrase thereof means administering a compound to the patient or causing the formation of a compound inside the body of the patient.

The pharmaceutical compounds of Formula (I) such as tris(8-quinolinolato)gallium(III) can be administered through intravenous injection or orally or any other suitable means at an amount of from 0.1 mg to 1000 mg per kg of body weight of the patient based on total body weight. The active ingredients may be administered at once, or may be divided into a number of smaller doses to be administered at predetermined intervals of time, e.g., once daily or once every two days. It should be understood that the dosage ranges set forth above are exemplary only and are not intended to limit the scope of this invention. The therapeutically effective amount of the active compound can vary with factors including, but not limited to, the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like. The amount of administration can be adjusted as the various factors change over time.

In accordance with the present invention, it is provided a use of a compound having a compound of Formula (I) or a pharmaceutically acceptable salt thereof (e.g., tris(8-quinolinolato)gallium(III)) for the manufacture of a medicament useful for treating a refractory cancer as described above. The medicament can be, e.g., in an oral or injectable form, e.g., suitable for intravenous, intradermal, or intramuscular administration. Injectable forms are generally known in the art, e.g., in buffered solution or suspension.

In accordance with another aspect of the present invention, a pharmaceutical kit is provided comprising in a container a unit dosage form of a compound of Formula (I) or a pharmaceutically acceptable salt thereof (e.g., tris(8-quinolinolato)gallium(III)), and optionally instructions for using the kit in the methods in accordance with the present invention, e.g., treating a previously treated cancer as described above, particularly a refractory or resistant cancer as described above, or preventing or delaying the recurrence of such a refractory cancer. As will be apparent to a skilled artisan, the amount of a therapeutic compound in the unit dosage form is determined by the dosage to be used on a patient in the methods of the present invention. In the kit, a compound having a compound of Formula (I) or a pharmaceutically acceptable salt thereof (e.g., tris(8-quinolinolato)gallium(III)) can be in a tablet form in an amount of, e.g., 1 mg.

### Example 1

### Activities of Tris(8-quinolinolato)gallium(III) in Colon Cancer Cell Line Resistant to Oxaliplatin or 5-FU

The colon carcinoma cell line HCT116 (HCT-116 p53 (+/+)) and the corresponding subline with deleted p53 genes (HCT-116 p53 (−/−)) were grown in McCoy's culture medium with 10% FCS. See Bunz et al., TARGETED INACTIVATION OF P53 IN HUMAN CELLS DOES NOT RESULT IN ANEUPLOIDY, CANCER RES., 62:1129-1133 (2002). For cytotoxicity assays, cells were plated (2×10³ cells in 100 μl/well) in 96-well plates and allowed to recover for 24 hours. Tris(8-quinolinolato)gallium(III) was added in another 100 μl growth medium and cells were exposed with the drug for 72 hours. The proportion of viable cells was determined by MTT assay following the manufacturer's recommendations (EZ4U, Biomedica, Vienna, Austria). The IC₅₀ values of tris(8-quinolinolato)gallium(III) (“Test Drug”) against the two different cell lines are provided in Table 1.

TABLE 1

| TUMOR | | |
|---|---|---|
| TYPE | CELL LINE | TEST DRUG IC50 (μM) |
| Colon | HCT-116 p53 (+/+) | 1.8 |
| Colon | HCT-116 p53 (−/−) | 1.7 |

The colon cancer cell line HCT-116 p53 (+/+) is sensitive to oxaliplatin treatment, while the colon cancer cell line HCT-116 p53 (−/−) is resistant to oxaliplatin. See e.g., Hata et al., Mol. Cancer. Ther., 4:1585 (2005). Therefore, tris(8-quinolinolato)gallium(III) is effective in inducing cell death in cells resistant to oxaliplatin.

In addition, it has been shown that colon cancer cell lines deficient in p53 are resistant to 5-fluorouracil treatment. See e.g., Bunz et al., J. Clin. Invest., 104:263-269 (1999); Bunz, et al., Science, 282:1497-1501 (1998). That is, the colon cancer cell line HCT-116 p53 (−/−) is resistant to 5-FU. Thus, the results in the above-described experiment also show that sodium tris(8-quinolinolato)gallium(III) is also effective in inducing cell death in cells resistant to 5-FU.

### Example 2

### Activities of Tris(8-quinolinolato)gallium(III) in Breast Cancer Cell Line Resistant to 5-FU

To test the activities of tris(8-quinolinolato)gallium(III) or 5-fluorouracil, ATCC's MTT Cell Proliferation Assay® was performed using human breast carcinoma cell line ZR-75-1. Stock cultures were allowed to grow to 70-80% confluence for this study. The anti-proliferative activity of tris(8-quinolinolato)gallium(III) or 5-fluorouracil against the indicated cell lines was evaluated in vitro using the ATCC's MTT Cell Proliferation Assay (Catalog No. 30-1010K). ZR-75-1 plates were seeded with 3,000 cells/well, and the cells were grown in RPMI1640 medium containing 1% (1M HEPES), 1% sodium pyruvate, 1% (45% Glucose), 10% FBS and 1% penicillin/strep/glutamine. Cultures were maintained in a 37° C. humidified 5% CO₂/95% air atmosphere. The cells were treated with tris(8-quinolinolato)gallium(III) or 5-fluorouracil at 1,000 μM, or a series of 4× dilutions thereof (250 μM, 62.5 μM, etc.). 100 μl of medium was removed from each well at 72 hours post-treatment and 10 μl MTT reagent was added to each well. The plates were incubated at 37° C. for 4 hours and then 100 μl of detergent was added. The plates were left overnight at room temperature in the dark and was read on a plate reader using SoftMax® Pro (version 5.2, Molecular Devices).

The absorbance data was analyzed as follows: Absorbance values were converted to Percent of Control and plotted against test agent concentrations for IC₅₀ calculations using SoftMax® Pro (version 5.2, Molecular Devices). The plate blank signal average was subtracted from all wells prior to calculating the Percent of Control. Percent of Control values were calculated by dividing the absorbance values for each test well by the No Drug Control average (column 11 values; cells+vehicle control) and multiplying by 100. Plots of Compound Concentration versus Percent of Control were analyzed using the 4-parameter equation to obtain IC₅₀ values and other parameters that describe the sigmoidal dose response curve.

The IC₅₀ value for the test agent was estimated by curve-fitting the data using the following four parameter-logistic equation:

$$Y = \frac{Top - Bottom}{1 + {( X / {IC}_{50} )}^{n}} + Bottom$$

wherein “Top” is the maximal % of control absorbance (100%), “Bottom” is the minimal % of control absorbance at the highest agent concentration (down to zero), Y is the Percent of Control absorbance, X is the test agent Concentration, IC₅₀ is the concentration of agent that inhibits cell growth by 50% compared to the control cells, n is the slope of the curve. Another human breast cancer cell line MX-1 was also tested in the same manner as described above to obtain IC₅₀ values of tris(8-quinolinolato)gallium(III) (“Test Drug”) and 5-FU. Table 2 summarizes the results.

TABLE 2

| | Test Drug | | 5-FU | |
|---|---|---|---|---|
| | | IC₅₀ Ratio | | IC₅₀ Ratio |
| Cell Line | IC₅₀ | (ZR-75-1/MX-1) | IC₅₀ | (ZR-75-1/MX-1) |
| ZR-75-1 | 1.59 μM | 0.77 | 31.1 μM | 21.2 |
| MX-1 | 2.06 μM | | 1.47 μM | |

Thus, tris(8-quinolinolato)gallium(III) is even more active in the ZR-75-1 cells which are significantly resistant to 5-FU.

In a separate experiment, tris(8-quinolinolato)gallium(III) and cisplatin were tested in breast cancer cell lines MCF-7 and MDA-MB-231 in the same manner as described above. Table 3 shows that tris(8-quinolinolato)gallium(III) is even more active in cells less sensitive to cisplatin.

TABLE 3

| | Test Drug | | Cisplatin | |
|---|---|---|---|---|
| | | IC₅₀ Ratio | | IC₅₀ Ratio |
| | | (MDA-MB-231/ | | (MDA-MB-231/ |
| Cell Line | IC₅₀ | MCF-7) | IC₅₀ | MCF-7) |
| MDA- | 0.838 μM | 0.681 | 47.1 μM | 5.88 |
| MB-231 | | | | |
| MCF-7 | 1.23 μM | | 8.01 μM | |

### Example 3

### Activities of Tris(8-quinolinolato)gallium(III) in BCRP-Overexpressing Breast Cancer Cells

Breast cancer cell line MDA-MB-231 and its corresponding bcrp-overexpressing line MDA-MB-231/bcrp were plated (2×10³ cells in 100 μl/well) in 96-well plates and allowed to recover for 24 hours. Tris(8-quinolinolato)gallium(III) was added in another 100 μl growth medium and were exposed to the drug for 72 hours. The proportion of viable cells was determined by MTT assay following the manufacturer's recommendations (EZ4U, Biomedica, Vienna, Austria), and IC₅₀ values were calculated. As shown in Table 4 below, tris(8-quinolinolato)gallium(III) is equally effective in BCRP-overexpressing cells. BCRP-overexpression confers resistance to drugs such as irinotecan, topotecan, mitoxantrone, methotrexate, camptothecins, gefitinib and imatinib etc. See Fojo & Menefee, Ann. Oncol., 18 (Supplement 5):v3-v8 (2007). Thus, tris(8-quinolinolato)gallium(III) should be effective in cells resistant to such drugs.

TABLE 4

| | tris(8-quinolinolato)gallium(III) |
|---|---|
| Cell Line | IC50 (μM) |
| MDA-MB-231 | 1.6 |
| MDA-MB-231/bcrp | 1.8 |

### Example 4

### Activities of Tris(8-quinolinolato)gallium(III) in Human Breast Cancer Cell Lines Overexpressing PGP

The alveolar epithelial cell carcinoma cell line SW-1573 and its MVP- and MRP1-overexpressing subline SW-1573/2R120 were grown in DMEM medium supplemented with 10% fetal calf serum. See Elbling et al., Cytometry, 31:187-198 (1998). Cells were plated (2×10³ cells in 100 μl/well) in 96-well plates and allowed to recover for 24 hours. Tris(8-quinolinolato)gallium(III) was added in another 100 μA growth medium and cells were exposed to the drug for 72 hours. The proportion of viable cells was determined by MTT assay following the manufacturer's recommendations (EZ4U, Biomedica, Vienna, Austria). Tris(8-quinolinolato)gallium(III) had substantially same IC₅₀ values in SW1573 cell line and its corresponding PGP/MRP-1-overexpressing cell line (1.7 μM and 2.1 μM, respectively). PGP/MRP-1-overexpression confers resistance to drugs such as vincristine, vinblastine, vinorelbine, taxol, docetaxel, etoposide, mitoxantrone, doxorubincin, epirubicin, topotecan, irinotecan, methotrexate, and imatinib etc. See Fojo & Menefee, Ann. Oncol., 18 (Supplement 5):v3-v8 (2007). Thus, tris(8-quinolinolato)gallium(III) should be effective in breast cancer cells resistant to such drugs.

### Example 5

### Activities of Tris(8-quinolinolato)gallium(III) in Human Lung Cancer Cell Lines Resistant to Cisplatin, Paclitaxel, Erlotinib or Gefitinib

Tris(8-quinolinolato)gallium(III), cisplatin and paclitaxel were tested in human lung carcinoma cell lines A549, NCI-H322M and H1975 in the same manner as described in Example 2 above to obtain their IC₅₀ values in the cell lines. Tables 5 and 6 below show that tris(8-quinolinolato)gallium(III) is almost equally effective in killing tumor cells that exhibit relative resistance to cisplatin or paclitaxel.

TABLE 5

| | Test Drug | | Paclitaxel | |
|---|---|---|---|---|
| | | IC₅₀ Ratio | | IC₅₀ Ratio |
| Cell Line | IC₅₀ | (A549/H1975) | IC₅₀ | (A549/H1975) |
| A549 | 1.11 μM | 0.31 | 9.92 μM | 1984 |
| H1975 | 3.55 μM | | 0.005 μM | |

TABLE 6

| | Test Drug | | cisplatin | |
|---|---|---|---|---|
| Cell | | IC₅₀ Ratio | | IC₅₀ Ratio |
| Line | IC₅₀ | (A549/H1975) | IC₅₀ | (NCI-H322M/H1975) |
| NCI- | 4.95 μM | 1.39 | 52.3 μM | 4.59 |
| H322M | | | | |
| H1975 | 3.55 μM | | 11.4 μM | |

In addition, it is known in the art that the human lung carcinoma cell line H 1975 is resistant to erlotinib and gefitinib due to the T790M mutation in the EGFR gene in the cells. See e.g., Bao et al., Mol. Cancer. Ther., 8(12):3296-3306 (2009). Thus, tris(8-quinolinolato)gallium(III) is also active against tumor cells (e.g., NSCLC cells) resistant to an EGFR inhibitor such as erlotinib and gefitinib.

Furthermore, as shown in Table 7 below, tris(8-quinolinolato)gallium(III) is more effective in NSCLC cells with KRAS mutation than those with wild-type KRAS. Thus, tris(8-quinolinolato)gallium(III) can be active against tumor cells (e.g., NSCLC cells) having an activating mutation in the KRAS gene, which are, e.g., typically resistant to EGFR inhibitors such as erlotinib and gefitinib, and EGFR antibodies.

TABLE 7

| Cell Line | KRAS status | IC₅₀ |
|---|---|---|
| A549 | G12S(GGT > AGT) | 1.11 μM |
| H1975 | Wild Type | 3.55 μM |
| NCI-H322 | Wild Type | 4.95 μM |

### Example 6

### Activities of Tris(8-quinolinolato)gallium(III) in Human Melanoma Cell Line

Tris(8-quinolinolato)gallium(III) and temozolomide were tested in human melanoma cell line A375 in the same manner as described in Example 2 above to obtain their IC₅₀ values in the cell line. The IC₅₀ of tris(8-quinolinolato)gallium(III) in the A375 cell line was 1.95 μM whereas the IC₅₀ of temozolomide was 369 μM.

### Example 7

### Activities of Tris(8-quinolinolato)gallium(III) in Human Prostate Cancer Cell Lines

Tris(8-quinolinolato)gallium(III) and were tested in human prostate cancer cell lines LNCaP (prostate carcinoma) and PC-3 (prostate carcinoma) in the same manner as described in Example 2 above to obtain their IC₅₀ values in the cell lines. In addition, the IC₅₀ values of docetaxel in the LNCaP and PC-3 cell lines were obtained from published literature. The ratios of IC₅₀ values of tris(8-quinolinolato)gallium(III) in a cell line sensitive to one of the other drugs and a cell line insensitive to the same drug were calculated. The results are shown in Table 8 below (“Test Drug” in the table denotes tris(8-quinolinolato)gallium(III)). The data shows that tris(8-quinolinolato)gallium(III) is in fact more effective in PC-3 cells which are resistant to docetaxel, paclitaxel, and vinblastine than in LNCaP cells that are relatively sensitive to docetaxel and vinblastine. See Domingo-Domenech et al., Clin. Cancer Res., 12(18):5578-5586 (2006); Blagosklonny et al., J. Urol., 163(3):1022-6 (2000). Therefore, tris(8-quinolinolato)gallium(III) is potentially effective in treating cancer resistant to such drugs.

TABLE 8

| | Test Drug | | Docetaxel* | |
|---|---|---|---|---|
| | | IC₅₀ Ratio | | IC₅₀ Ratio |
| Cell Line | IC₅₀ | (PC-3/LNCaP) | IC₅₀ | (PC-3/LNCaP/) |
| PC-3 | 1.79 μM | 0.82 | 50-60 nM | 12-20 |
| LNCaP | 2.19 μM | | 2.5-5 nM | |

*Domingo-Domenech et al., Clin. Cancer Res., 12(18): 5578-5586 (2006).

### Example 8

### Activity of Tris(8-quinolinolato)gallium(III) in Human Renal Cancer Cells Resistant to Temsirolimus

Tris(8-quinolinolato)gallium(III) was tested in human renal cell lines A498 in the same manner as described in Example 2 above to obtain its IC₅₀ value in the cell line. Tris(8-quinolinolato)gallium(III) was able to cause apoptosis in A498 cells (IC₅₀ is 32.9 μM). It is known in the art that A498 cells exhibit resistance to temsirolimus. See e.g., Mahalingam et al., Clin. Cancer Res., 16(1):141-153 (2010). Thus, tris(8-quinolinolato)gallium(III) may be useful in treating renal cancer resistant to temsirolimus.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be apparent that certain changes and modifications may be practiced within the scope of the appended claims.

1-9. (canceled)

10. A method of treating a cancer previously treated with a regimen comprising one or more drugs chosen from the group consisting of 5-FU, camptothecin, gefitinib, erlotinib, icotinib, temsirolimus and temozolomide, said method comprising administration of tris(8-quinolinolato)gallium(III), or a pharmaceutically acceptable salt thereof, to a subject in need thereof.

11. A method of treating a colorectal cancer previously treated with a regimen comprising one or both of oxaliplatin and 5-FU, said method comprising administration of tris(8-quinolinolato)gallium(III), or a pharmaceutically acceptable salt thereof, to a subject in need thereof.

12. A method of treating a melanoma previously treated with a regimen comprising temozolomide, said method comprising administration of tris(8-quinolinolato)gallium(III), or a pharmaceutically acceptable salt thereof, to a subject in need thereof.

13. A method of treating lung cancer previously treated with a regimen comprising one or more drugs chosen from paclitaxel, erlotinib, icotinib and gefitinib, said method comprising administration of tris(8-quinolinolato)gallium(III), or a pharmaceutically acceptable salt thereof, to a subject in need thereof.

14. A method of treating non-small cell lung cancer having an activating mutation in the KRAS gene, said method comprising administration of tris(8-quinolinolato)gallium(III), or a pharmaceutically acceptable salt thereof, to a subject in need thereof.
